(19) United States
(12) Patent Application Publication
(10) Pub. No.: US 2026/0157784 A1
(43) Pub. Date: Jun. 11, 2026

(54) MODEL-BASED DETERMINATION OF A SMOKE EVACUATION TIME WINDOW FOR USE DURING A MEDICAL SESSION

(71) Applicant: Intuitive Surgical Operations, Inc., Sunnyvale, CA (US)

(72) Inventors: Humphrey W. Chow, Cupertino, CA (US); Mingkai Hsueh, Milpitas, CA (US); Brandon D. Itkowitz, San Jose, CA (US)

(21) Appl. No.: 18/707,291

(22) PCT Filed: Nov. 11, 2022
(86) PCT No.: PCT/US2022/049714

§ 371 Date: May 3, 2024

Related U.S. Application Data
(60) Provisional application No. 63/279,352, filed on Nov. 15, 2021.

(51) Int. Cl. A61B 18/04; A61B 18/00

(52) U.S. Cl. CPC A61B 18/04; A61B 2018/00595; A61B 2218/008

(57) ABSTRACT

A smoke evacuation management system may include a memory storing instructions and a processor communicatively coupled to the memory. The processor may be configured to access a model generated based on data representative of one or more attributes associated with one or more energy application events during which energy is applied by one or more energy instruments to one or more subjects. The processor may be further configured to set, based on the model, a parameter of a smoke evacuation time window for use during a medical session in which an energy instrument applies intraoperative energy to a subject, the smoke evacuation time window specifying a time period during which a smoke evacuation procedure is performed. The processor may be further configured to direct, based on the smoke evacuation time window, a performance of the smoke evacuation procedure in response to the energy instrument applying the intraoperative energy.

### RELATED APPLICATIONS

The present application claims priority to U.S. Provisional Patent Application Ser. No. 63/279,352, filed Nov. 15, 2021, the contents of which is hereby incorporated by reference in its entirety.

### BACKGROUND INFORMATION

Smoke generated from use of energy (e.g., cautery energy) applied to tissue within a cavity of a patient (or any other type of subject) during a medical procedure may accumulate within the cavity and cause various problems. For example, the smoke may occlude vision, thereby hindering progress of the medical procedure. Furthermore, constituents of the smoke are toxic and may harm the subject if left to settle in place. Thus, smoke evacuation procedures are often applied to evacuate the smoke from the cavity. Such procedures often involve gas exchange, including suctioning and replenishing gas to remove smoke while maintaining pressure in the cavity. Unfortunately, such gas exchange may also cause a number of undesirable side-effects, such as desiccation of tissue and/or the removal of heat from the patient leading to hypothermia as the replenishing gas may be cold and dry. Hence, it is desirable to avoid subjecting the patient to more smoke evacuation than that which is necessary to adequately evacuate the smoke from the cavity.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An exemplary system includes a memory storing instructions and a processor communicatively coupled to the memory and configured to access a model generated based on data representative of one or more attributes associated with one or more energy application events during which energy is applied by one or more energy instruments to one or more subjects; set, based on the model, a parameter of a smoke evacuation time window for use during a medical session in which an energy instrument applies intraoperative energy to a subject, the smoke evacuation time window specifying a time period during which a smoke evacuation procedure is performed; and direct, based on the smoke evacuation time window, a performance of the smoke evacuation procedure in response to the energy instrument applying the intraoperative energy.

An exemplary method includes accessing a model generated based on data representative of one or more attributes associated with one or more energy application events during which energy is applied by one or more energy instruments to one or more subjects; setting, based on the model, a parameter of a smoke evacuation time window for use during a medical session in which an energy instrument applies intraoperative energy to a subject, the smoke evacuation time window specifying a time period during which a smoke evacuation procedure is performed; and directing, based on the smoke evacuation time window, a performance of the smoke evacuation procedure in response to the energy instrument applying the intraoperative energy.

An exemplary non-transitory computer-readable medium stores instructions executable by a processor to access a model generated based on data representative of one or more attributes associated with one or more energy application events during which energy is applied by one or more energy instruments to one or more subjects; set, based on the model, a parameter of a smoke evacuation time window for use during a medical session in which an energy instrument applies intraoperative energy to a subject, the smoke evacuation time window specifying a time period during which a smoke evacuation procedure is performed; and direct, based on the smoke evacuation time window, a performance of the smoke evacuation procedure in response to the energy instrument applying the intraoperative energy.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIGS. 1-2 depict an illustrative smoke evacuation management system according to principles described herein.

FIGS. 3-4 depict illustrative predictive models for smoke evacuation according to principles described herein.

FIG. 5 depicts an illustrative graph of smoke evacuation time windows according to principles described herein.

FIG. 6 depicts an illustrative computer-assisted robotic surgical system according to principles described herein.

FIG. 7 depicts an illustrative method according to principles described herein.

FIG. 8 depicts an illustrative computing device according to principles described herein.

### DETAILED DESCRIPTION

Systems and methods for surgical smoke evacuation are described herein. A smoke evacuation management system may access a model generated based on data representative of one or more attributes associated with one or more energy application events during which energy is applied by one or more energy instruments to one or more subjects. The system may set, based on the model, a parameter of a smoke evacuation time window for use during a medical session in which an energy instrument applies intraoperative energy to a subject, the smoke evacuation time window specifying a time period during which a smoke evacuation procedure is performed. The system may further direct, based on the smoke evacuation time window, a performance of the smoke evacuation procedure in response to the energy instrument applying the intraoperative energy.

During a medical procedure (e.g., a minimally-invasive surgical procedure), a user (e.g., a surgeon) may use an energy instrument to apply energy (also referred to herein as intraoperative energy) to a subject. For example, a surgeon may use a cautery tool to apply energy to cut tissue, cauterize tissue, coagulate tissue, etc. Such energy application events may result in smoke being generated. In a minimally-invasive surgery, the smoke may fill a cavity of the subject unless evacuated. Such smoke may be harmful to surrounding tissue in the cavity and/or occlude visibility for the surgeon and may interfere with a performance of the surgery. Thus, smoke may be evacuated by a smoke evacuation system, which may suction gas from within the cavity, thereby evacuating the smoke. The smoke evacuation system may also backfill gas into the cavity, insufflating the cavity to a defined pressure to maintain a minimally-invasive surgical space. While a maximizing of evacuation of smoke may be optimal for surgical efficiency, the gas exchange may have negative side effects for the subject such as desiccation of tissue or the removal of heat from the patient leading to hypothermia.

Systems and methods described herein may be configured to optimize a smoke evacuation time window that specifies a time period during which a smoke evacuation procedure is performed with respect to one or more energy application events. Such optimization may be based on a predictive model generated based on historical data of prior energy application events. The predictive model may be configured to predict an occurrence of a next energy application event and optimize the smoke evacuation time window for that energy application event based on such prediction.

Systems and methods described herein may provide various advantages and benefits. For example, systems and methods described herein may provide for an optimized smoke evacuation time window that allows for sufficient smoke evacuation without overly exposing the subject to gas exchange. The optimized smoke evacuation may allow the surgeon to efficiently perform the surgical procedure. As the smoke evacuation time window is set automatically based on the predictive model, the surgeon may progress through the surgical procedure without providing additional input for the smoke evacuation procedure and/or waiting for the smoke evacuation. The reduced gas exchange may minimize injury to tissue of the subject caused by the gas exchange of the smoke evacuation. Thus systems and methods described herein may allow for medical procedures to be performed more efficiently and safely than conventional systems.

Various illustrative embodiments will now be described in more detail. The disclosed systems and methods may provide one or more of the benefits mentioned above and/or various additional and/or alternative benefits that will be made apparent herein.

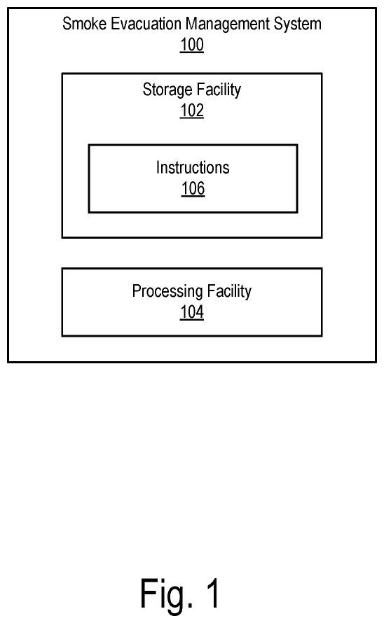

FIG. 1 illustrates an exemplary smoke evacuation management system 100 (“system 100”) configured to perform various operations described herein. As shown, system 100 may include, without limitation, a storage facility 102 and a processing facility 104 selectively and communicatively coupled to one another. Facilities 102 and 104 may each include or be implemented by hardware and/or software components (e.g., processors, memories, communication interfaces, instructions stored in memory for execution by the processors, etc.). For example, facilities 102 and/or 104 may be implemented by any component in a computer-assisted medical system configured to perform a medical procedure. As another example, facilities 102 and/or 104 may be implemented by a computing device separate from and communicatively coupled to a computer-assisted medical system. Although facilities 102 and 104 are shown to be separate facilities in FIG. 1, facilities 102 and 104 may be combined into fewer facilities, such as into a single facility, or divided into more facilities as may serve a particular implementation. In some examples, each of facilities 102 and 104 may be distributed between multiple devices and/or multiple locations as may serve a particular implementation.

Storage facility 102 may maintain (e.g., store) executable data used by processing facility 104 to perform one or more of the operations described herein. For example, storage facility 102 may store instructions 106 that may be executed by processing facility 104 to perform one or more of the operations described herein. Instructions 106 may be implemented by any suitable application, software, code, and/or other executable data instance. Storage facility 102 may also maintain any data received, generated, managed, used, and/or transmitted by processing facility 104.

Processing facility 104 may be configured to perform (e.g., execute instructions 106 stored in storage facility 102 to perform) various operations described herein. For example, processing facility 104 may be configured to access a predictive model configured to predict energy application events in a medical session and set, based on the predictive model, a parameter of a smoke evacuation time window for evacuating smoke generated by the energy application events.

These and other operations that may be performed by system 100 (e.g., processing facility 104) are described herein.

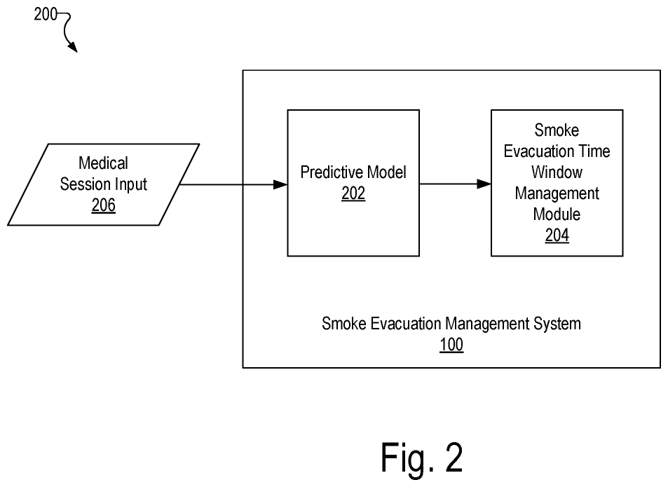

FIG. 2 illustrates an example configuration 200 of smoke evacuation management system 100. Configuration 200 shows system 100 including a predictive model 202 and a smoke evacuation time window management module 204 (“module 204”). Predictive model 202 receives medical session input 206 and based on output from predictive model 202, module 204 may set one or more parameters of a smoke evacuation time window for a medical session.

A medical session may include any suitable medical procedure and any activities associated with the medical procedure, such as pre-procedure activities (e.g., setup activities), intra-procedure activities, and/or post-procedure activities. The medical procedure may include any activity conducted on a subject, such as minimally-invasive surgical procedures, open surgical procedures, non-surgical procedures, diagnostic procedures, therapeutic procedures, procedures in clinical, non-clinical, and/or training settings, etc. A subject may include any person or part of a person, body, or object on which a medical procedure may be performed, such as a body of a live animal, a human or animal cadaver, a portion of human or animal anatomy, tissue removed from human or animal anatomies, non-tissue work pieces, training models, etc.

Predictive model 202 may include a model generated using any suitable algorithm or algorithms based on data (e.g., statistical data) associated with energy application events. Such statistical data may be from one or more previous medical sessions prior to a current medical session and/or from energy application events that occur earlier in the current medical session. Such statistical data may also be categorized based upon specific users, procedure types, energy devices used, etc. that may then be strategically grouped to form specific model outputs optimal for the current medical session. Based on the statistical data and medical session input 206, predictive model 202 may be configured to predict a likelihood of future energy application events occurring during the current medical session. Based on such predictions, module 204 may set one or more parameters of a smoke evacuation time window for the current medical session. Examples of predictive model 202 are further described herein.

Module 204 may be configured to access output from predictive model 202 to determine parameters of the smoke evacuation time window. Module 204 may set any suitable parameters of the smoke evacuation time window, such as a duration of the time period of the smoke evacuation time window, a start time with respect to an energy application event of the medical session, or an end time with respect to an energy application event of the medical session. Module 204 may further be configured to set any suitable parameters associated with the smoke evacuation procedure, such as an air flow parameter associated with an air flow of the smoke evacuation procedure (e.g., how quickly gas is exchanged), a location within the subject at which the smoke evacuation procedure is performed (e.g., a location where gas is suctioned and/or a location where gas is insufflated), and/or a technique used to perform the smoke evacuation procedure. The smoke evacuation procedure may include any suitable technique that results in an evacuation of smoke from within the subject. Examples of setting parameters are further described herein.

Medical session input 206 may include any suitable input accessed (e.g., detected, determined, received, retrieved, generated, etc.) during the medical session. For example, medical session input 206 may include data representative of attributes corresponding to the attributes used for generating predictive model 202. For instance, predictive model 202 may be generated based at least in part on data representative of a time period between energy application events. Correspondingly, medical session input 206 may include data representative of time periods between energy application events occurring during the current medical session. Such medical session input 206 may be used as an input to predictive model 202 so that predictive model 202 may generate predictions of future energy application events based on user behavior and/or other variables of the current medical session.

Medical session input 206 may further include user input associated with a user input device that is configured to control an operation of an energy instrument. For example, the energy instrument may be activated by the user input device (e.g., a foot pedal, a button, etc.). Medical session input 206 may include data representative of user interaction relative to the user input device, such as a user approaching the user input device (e.g., a foot of the user hovering over a foot pedal included in a computer-assisted medical system), a degree of actuation of the user input device (e.g., a light or partial press of a foot pedal or button, etc.), a type of interaction with the user input device (e.g., a single press, a double press, a press and hold, etc.), etc. Such data may be included as additional bases for predicting energy activation events and module 204 may set parameters of the smoke evacuation time window accordingly. Such user input may be detected in any suitable manner, such as by one or more sensors (e.g., proximity sensors, motion sensors, etc.) included on or near the user input device, image and/or video analysis of images captured by one or more imaging devices, the user input device, an additional user input device (e.g., a voice activated input device, an additional button or pedal, etc.), etc. Furthermore, medical session input 206 may include information associated with the type of medical session, the type of electrosurgical energy device or instrument being used, the device settings, and/or the user of the energy instrument, etc.

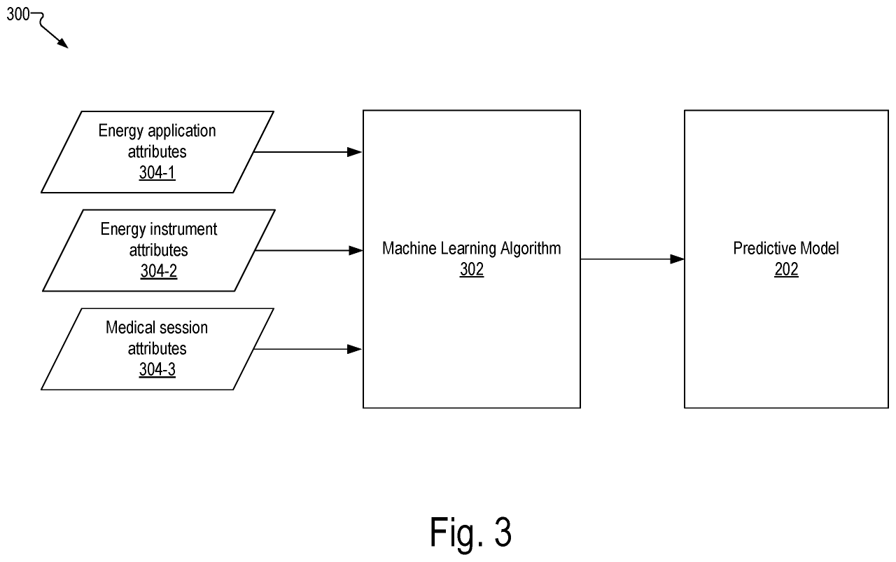

FIG. 3 illustrates an example configuration 300 of generating predictive model 202. Configuration 300 includes a machine learning algorithm 302 that accesses data representative of attributes 304 to generate predictive model 202 based on the data. Machine learning algorithm 302 may include any suitable one or more machine learning algorithms as well as non-machine learning algorithms configured to analyze data and generate a model configured to predict a likelihood of future events. For instance, machine learning algorithm 302 may include a multiple regression analysis of attributes 304.

Attributes 304 may be associated with one or more energy application events during which energy is applied by one or more energy instruments to one or more subjects (e.g., a subject of a current medical session and/or one or more subjects of previous medical sessions). Attributes 304 may include any suitable characteristics associated with the one or more energy application events that may provide correlative and/or predictive value to the one or more energy application events and/or future energy application events. For instance, attributes 304 may include energy application attributes 304-1, such as a frequency of the one or more energy application events, a number of the one or more energy application events (e.g., a count of applications in a particular medical session), a duration of the one or more energy application events, a time period between energy application events included in the one or more energy application events, a type of energy application of the one or more energy application events (e.g., a modality of energy application such as cutting, cautery, etc.), an amount of energy applied by the one or more application events (e.g., a wattage), etc.

For example, energy application events may tend to be grouped together such that the time period between energy application events may be highly indicative of a likelihood of another energy application event. Consequently, if the time period extends beyond a particular predetermined threshold time period then the likelihood of another energy application event may fall to below a predetermined threshold likelihood so that a smoke evacuation time window management module may close the smoke evacuation time window and deactivate a smoke evacuation procedure. Similarly, other energy application attributes 304-1 (or combinations of energy application attributes 304-1 and/or attributes 304) may be found to be indicative of a likelihood of future energy application events.

Attributes 304 may further include energy instrument attributes 304-2 (e.g., a type of the one or more energy instruments, energy application characteristics such as monopolar or bipolar, etc.) and medical session attributes 304-3. Medical session attributes 304-3 may include any suitable attributes associated with the medical session, such as a type of medical procedure (e.g., urologic, gynecologic, etc.), a phase of the medical session, attributes associated with one or more users of the one or more energy instruments (e.g., an energy application tendency of a particular surgeon or group of surgeons), attributes associated with the one or more subjects (e.g., a sensitivity to smoke versus carbon dioxide or other insufflating gas of a particular subject or group of subjects, a type of location and/or tissue within a subject exposed to the smoke evacuation procedure, etc.), user inputs relative to a user input device configured to control an operation of the energy instrument, etc.

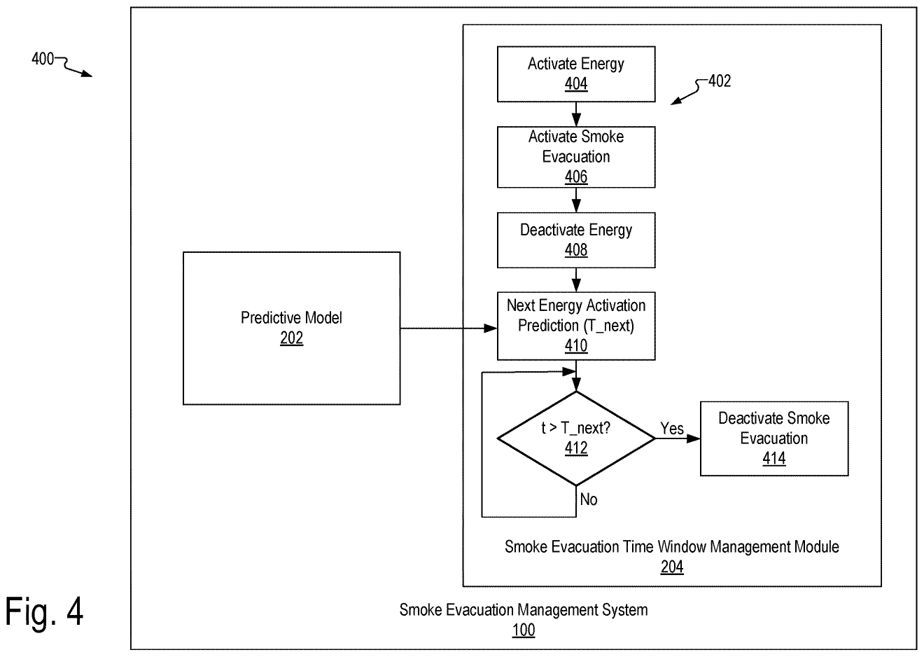

FIG. 4 illustrates another example configuration 400 of smoke evacuation management system 100 including predictive model 202 and an implementation for smoke evacuation management module 204 applying a procedure represented by flow chart 402.

At block 404, an activation of an energy instrument to apply energy to a subject during a medical session may be detected. At block 406, module 204 may start a smoke evacuation time window, which activates a smoke evacuation procedure. At block 408, a deactivation of the energy instrument may be detected. A time of the deactivation (T_off) may be stored. At block 410, module 204 may access an output of predictive model 202 that provides a prediction of a next energy instrument activation time (T_next). At block 412, module 204 may compare a current time (t) with the predicted next energy instrument activation time (t>T_next). If the current time has not yet reached the predicted next energy instrument activation time, module 204 may return to block 412, looping until t>T_next and keeping the smoke evacuation time window open. During this time, if module 204 detects another energy instrument activation, module 204 may restart the process of flow chart 402. If the current time passes the predicted next energy activation time without another energy instrument activation, at block 414, module 204 may end the smoke evacuation time window and deactivate the smoke evacuation procedure.

In some examples, a starting of the smoke evacuation time window may be configured to precede an energy instrument activation of an energy application event. As a location of the smoke evacuation procedure may be slightly removed from a location of the energy activation event so that a user is not obstructed by the smoke evacuation procedure, there may be a delay between a starting of the smoke evacuation procedure and a starting of an evacuation of smoke. Thus, module 204 may be configured to start the smoke evacuation procedure prior to (e.g., several seconds before) the energy instrument activation so that smoke generated by the energy application may be evacuated as the smoke starts being generated. Such a starting of the smoke evacuation procedure prior to the energy application event may be based on the prediction of the energy application event as provided by predictive model 202.

In some instances, the smoke evacuation procedure may have already been started for a previous energy application event, and thus module 204 may continue the smoke evacuation procedure in anticipation of a predicted next energy application event (e.g., as illustrated in flow chart 402). Additionally or alternatively, predictive model 202 may predict an initial energy application event (or an energy application event after a time period without energy application events). In some examples, such predictions of initial energy application events may be based on a particular subset of attributes associated with one or more previous energy application events and/or a particular weighting of the attributes. For example, predictive model 202 may weigh more heavily user input relative to the user input device for predicting initial energy application events as opposed to subsequent energy application events. Any other such weighting may be used that provides predictive value based on the statistical data.

In some examples, predictions provided by predictive model 202 may include a predicted activation time of a next energy application event (e.g., as illustrated in flow chart 402). Additionally or alternatively, predictions may include a likelihood (e.g., a percentage) of a next energy application event at a given time. Module 204 may be configured to set parameters of the smoke evacuation time window based on such outputs in any suitable manner.

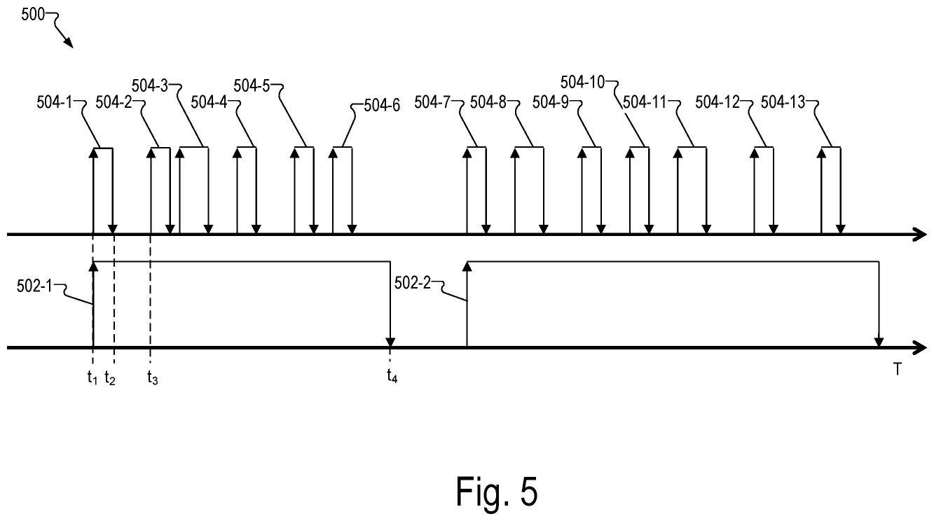

FIG. 5 illustrates an example graph 500 showing smoke evacuation time windows 502 (e.g., smoke evacuation time window 502-1 and 502-2) relative to energy application events 504 (e.g., energy application events 504-1 through 504-13) across time (T). In example graph 500, parameters of smoke evacuation time windows 502 may be set based on the procedure represented by flow chart 402.

For instance, smoke evacuation management system 100 may detect at time t₁ an energy instrument activation for an initial energy application event 504-1. Based on the detection, system 100 may start smoke evacuation time window 502-1. System 100 may then detect at time t₂ an energy instrument deactivation for energy application event 504-1. System 100 may determine, based on a predictive model (e.g., predictive model 202) a time (T_next) for an occurrence of a next energy activation event (e.g., t₂+a predicted time period, such as 10 seconds). At t₃, system 100 may detect an energy instrument activation for a second energy application event 504-2. As t₃ occurs before the predicted time T_next, system 100 may keep smoke evacuation time window 502-1 open. In this example, this process may repeat until energy application event 504-6, after which another energy application event is not detected before the next predicted energy application event time. At that point (time t₄), system 100 may close smoke evacuation time window 502-1.

In this manner, the smoke evacuation procedure may already be running for energy application events 504-2 through 504-6, which may allow for optimized smoke removal compared to restarting the smoke evacuation procedure for each energy application event. Furthermore, closing the smoke evacuation time window after energy application event 504-6 may reduce exposure of the subject to gas exchange and attendant side effects.

As shown, on detection of another energy application event 504-7 after a time period without energy application events, system 100 may open another smoke evacuation time window 502-2 and repeat the process across energy application events 504-7 through 504-13.

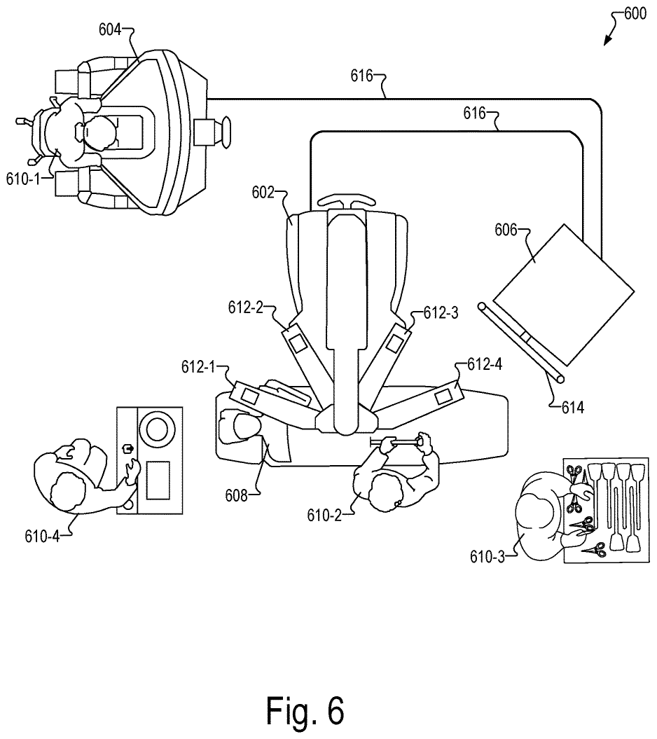

As has been described, system 100 may be associated in certain examples with a computer-assisted medical system used to perform a medical procedure on a subject. To illustrate, FIG. 6 shows an illustrative computer-assisted medical system 600 that may be used to perform various types of medical procedures including surgical and/or non-surgical procedures.

As shown, computer-assisted medical system 600 may include a manipulator assembly 602 (a manipulator cart is shown in FIG. 6), a user control apparatus 604, and an auxiliary apparatus 606, all of which are communicatively coupled to each other. Computer-assisted medical system 600 may be utilized by a medical team to perform a computer-assisted medical procedure or other similar operation on a body of a subject 608 or on any other body as may serve a particular implementation. As shown, the medical team may include a first user 610-1 (such as a surgeon for a surgical procedure), a second user 610-2 (such as a subject-side assistant), a third user 610-3 (such as another assistant, a nurse, a trainee, etc.), and a fourth user 610-4 (such as an anesthesiologist for a surgical procedure), all of whom may be collectively referred to as users 610, and each of whom may control, interact with, or otherwise be a user of computer-assisted medical system 600. More, fewer, or alternative users may be present during a medical procedure as may serve a particular implementation. For example, team composition for different medical procedures, or for non-medical procedures, may differ and include users with different roles.

While FIG. 6 illustrates an ongoing minimally invasive medical procedure such as a minimally invasive surgical procedure, it will be understood that computer-assisted medical system 600 may similarly be used to perform open medical procedures or other types of operations. For example, operations such as exploratory imaging operations, mock medical procedures used for training purposes, and/or other operations may also be performed.

As shown in FIG. 6, manipulator assembly 602 may include one or more manipulator arms 612 (e.g., manipulator arms 612-1 through 612-4) to which one or more instruments may be coupled. The instruments may be used for a computer-assisted medical procedure on subject 608 (e.g., in a surgical example, by being at least partially inserted into subject 608 and manipulated within subject 608). While manipulator assembly 602 is depicted and described herein as including four manipulator arms 612, it will be recognized that manipulator assembly 602 may include a single manipulator arm 612 or any other number of manipulator arms as may serve a particular implementation. While the example of FIG. 6 illustrates manipulator arms 612 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 612 shown in FIG. 6.

During the medical operation, user control apparatus 604 may be configured to facilitate teleoperational control by user 610-1 of manipulator arms 612 and instruments attached to manipulator arms 612. To this end, user control apparatus 604 may provide user 610-1 with imagery of an operational area associated with subject 608 as captured by an imaging device. To facilitate control of instruments, user control apparatus 604 may include a set of master controls. These master controls may be manipulated by user 610-1 to control movement of the manipulator arms 612 or any instruments coupled to manipulator arms 612.

Auxiliary apparatus 606 may include one or more computing devices configured to perform auxiliary functions in support of the medical procedure, such as providing insufflation, electrocautery energy, illumination or other energy for imaging devices, image processing, or coordinating components of computer-assisted medical system 600. In some examples, auxiliary apparatus 606 may be configured with a display monitor 614 configured to display one or more user interfaces, or graphical or textual information in support of the medical procedure. In some instances, display monitor 614 may be implemented by a touchscreen display and provide user input functionality. Augmented content provided by a region-based augmentation system may be similar, or differ from, content associated with display monitor 614 or one or more display devices in the operation area (not shown).

Manipulator assembly 602, user control apparatus 604, and auxiliary apparatus 606 may be communicatively coupled one to another in any suitable manner. For example, as shown in FIG. 6, manipulator assembly 602, user control apparatus 604, and auxiliary apparatus 606 may be communicatively coupled by way of control lines 616, which may represent any wired or wireless communication link as may serve a particular implementation. To this end, manipulator assembly 602, user control apparatus 604, and auxiliary apparatus 606 may each include one or more wired or wireless communication interfaces, such as one or more local area network interfaces, Wi-Fi network interfaces, cellular interfaces, and so forth.

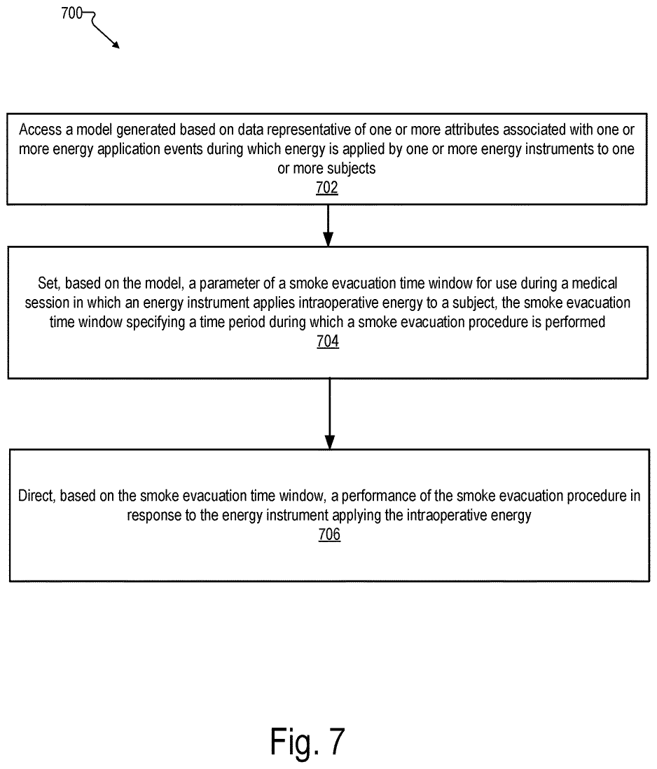

FIG. 7 illustrates an exemplary method 700 of a smoke evacuation management system. While FIG. 7 illustrates exemplary operations according to one embodiment, other embodiments may omit, add to, reorder, combine, and/or modify any of the operations shown in FIG. 7. One or more of the operations shown in in FIG. 7 may be performed by an smoke evacuation management system such as system 100, any components included therein, and/or any implementation thereof.

At operation 702, a smoke evacuation management system may access a model generated based on data representative of one or more attributes associated with one or more energy application events during which energy is applied by one or more energy instruments to one or more subjects. Operation 702 may be performed in any of the ways described herein.

At operation 704, the smoke evacuation management system may set, based on the model, a parameter of a smoke evacuation time window for use during a medical session in which an energy instrument applies intraoperative energy to a subject, the smoke evacuation time window specifying a time period during which a smoke evacuation procedure is performed. Operation 704 may be performed in any of the ways described herein.

At operation 706, the smoke evacuation management system may direct, based on the smoke evacuation time window, a performance of the smoke evacuation procedure in response to the energy instrument applying the intraoperative energy.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

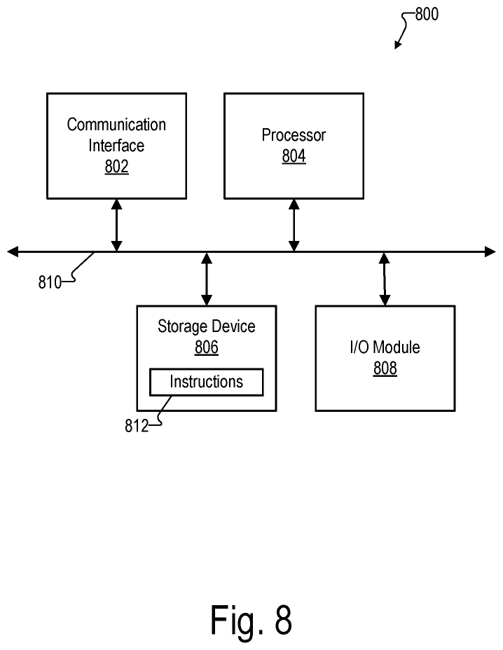

FIG. 8 illustrates an exemplary computing device 800 that may be specifically configured to perform one or more of the processes described herein. Any of the systems, units, computing devices, and/or other components described herein may implement or be implemented by computing device 800.

As shown in FIG. 8, computing device 800 may include a communication interface 802, a processor 804, a storage device 806, and an input/output (“I/O”) module 808 communicatively connected one to another via a communication infrastructure 810. While an exemplary computing device 800 is shown in FIG. 8, the components illustrated in FIG. 8 are not intended to be limiting. Additional or alternative components may be used in other embodiments. Components of computing device 800 shown in FIG. 8 will now be described in additional detail.

Communication interface 802 may be configured to communicate with one or more computing devices. Examples of communication interface 802 include, without limitation, a wired network interface (such as a network interface card), a wireless network interface (such as a wireless network interface card), a modem, an audio/video connection, and any other suitable interface.

Processor 804 generally represents any type or form of processing unit capable of processing data and/or interpreting, executing, and/or directing execution of one or more of the instructions, processes, and/or operations described herein. Processor 804 may perform operations by executing computer-executable instructions 812 (e.g., an application, software, code, and/or other executable data instance) stored in storage device 806.

Storage device 806 may include one or more data storage media, devices, or configurations and may employ any type, form, and combination of data storage media and/or device. For example, storage device 806 may include, but is not limited to, any combination of the non-volatile media and/or volatile media described herein. Electronic data, including data described herein, may be temporarily and/or permanently stored in storage device 806. For example, data representative of computer-executable instructions 812 configured to direct processor 804 to perform any of the operations described herein may be stored within storage device 806. In some examples, data may be arranged in one or more databases residing within storage device 806.

I/O module 808 may include one or more I/O modules configured to receive user input and provide user output. I/O module 808 may include any hardware, firmware, software, or combination thereof supportive of input and output capabilities. For example, I/O module 808 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 808 is configured to provide graphical data to a display for presentation to a user. The graphical data may be representative of one or more graphical user interfaces and/or any other graphical content as may serve a particular implementation.

In some examples, any of the systems, modules, and/or facilities described herein may be implemented by or within one or more components of computing device 800. For example, one or more applications 812 residing within storage device 806 may be configured to direct an implementation of processor 804 to perform one or more operations or functions associated with processing facility 104 of system 100.

As mentioned, one or more operations described herein may be performed during a medical procedure, e.g., dynamically, in real time, and/or in near real time. As used herein, operations that are described as occurring “in real time” will be understood to be performed immediately and without undue delay, even if it is not possible for there to be absolutely zero delay.

Any of the systems, devices, and/or components thereof may be implemented in any suitable combination or sub-combination. For example, any of the systems, devices, and/or components thereof may be implemented as an apparatus configured to perform one or more of the operations described herein.

In the description herein, various exemplary embodiments have been described. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
access a model generated based on data representative of one or more attributes associated with one or more energy application events during which energy is applied by one or more energy instruments to one or more subjects;
set, based on the model, a parameter of a smoke evacuation time window for use during a medical session in which an energy instrument applies intraoperative energy to a subject, the smoke evacuation time window specifying a time period during which a smoke evacuation procedure is performed; and
direct, based on the smoke evacuation time window, a performance of the smoke evacuation procedure in response to the energy instrument applying the intraoperative energy.

2. The system of claim 1, wherein the one or more energy application events comprise an energy application event from a previous medical session prior to the medical session.

3. The system of claim 1, wherein the one or more energy application events comprise an energy application that occurs during the medical session.

4. The system of claim 1, wherein the one or more attributes comprise at least one of a frequency of the one or more energy application events, a duration of the one or more energy application events, or a time period between energy application events included in the one or more energy application events.

5. The system of claim 1, wherein the one or more attributes comprise at least one of a type of the one or more energy instruments, a type of energy application of the one or more energy application events, or an amount of energy applied by the one or more energy application events.

6. The system of claim 1, wherein the one or more attributes comprise at least one of a phase of a medical session associated with the one or more energy application events or a type of a medical procedure associated with the one or more energy application events.

7. The system of claim 1, wherein the one or more attributes comprise one or more attributes associated with a user of the one or more energy instruments.

8. The system of claim 1, wherein the one or more attributes comprise one or more attributes associated with the one or more subjects.

9. The system of claim 1, wherein the setting the parameter of the smoke evacuation time window comprises setting a duration of the time period.

10. The system of claim 1, wherein the setting the parameter of the smoke evacuation time window comprises setting at least one of:
a start time with respect to an energy application event of the medical session, or
an end time with respect to the energy application event of the medical session.

11. The system of claim 1, wherein the processor is further configured to execute the instructions to set, based on the model, at least one of:
an air flow parameter associated with an air flow of the smoke evacuation procedure,
a location within the subject at which the smoke evacuation procedure is performed, or
a technique used to perform the smoke evacuation procedure.

12. The system of claim 1, wherein the setting the parameter of the smoke evacuation time window is further based on user input provided during the medical session.

13. The system of claim 12, wherein the user input is associated with a user input device configured to control an operation of the energy instrument.

14. A method comprising:
accessing a model generated based on data representative of one or more attributes associated with one or more energy application events during which energy is applied by one or more energy instruments to one or more subjects;
setting, based on the model, a parameter of a smoke evacuation time window for use during a medical session in which an energy instrument applies intraoperative energy to a subject, the smoke evacuation time window specifying a time period during which a smoke evacuation procedure is performed; and
directing, based on the smoke evacuation time window, a performance of the smoke evacuation procedure in response to the energy instrument applying the intraoperative energy.

15. The method of claim 14, wherein the one or more energy application events comprise an energy application event from a previous medical session prior to the medical session.

16. The method of claim 14, wherein the one or more energy application events comprise an energy application that occurs during the medical session.

17. The method of claim 14, wherein the one or more attributes comprise at least one of a frequency of the one or more energy application events, a duration of the one or more energy application events, or a time period between energy application events included in the one or more energy application events.

18. The method of claim 14, wherein the one or more attributes comprise at least one of a type of the one or more energy instruments, a type of energy application of the one or more energy application events, or an amount of energy applied by the one or more energy application events.

19. The method of claim 14, wherein the one or more attributes comprise at least one of a phase of a medical session associated with the one or more energy application events or a type of a medical procedure associated with the one or more energy application events.

20-26. (canceled)

27. A non-transitory computer-readable medium storing instructions executable by a processor to:
access a model generated based on data representative of one or more attributes associated with one or more energy application events during which energy is applied by one or more energy instruments to one or more subjects;
set, based on the model, a parameter of a smoke evacuation time window for use during a medical session in which an energy instrument applies intraoperative energy to a subject, the smoke evacuation time window specifying a time period during which a smoke evacuation procedure is performed; and
direct, based on the smoke evacuation time window, a performance of the smoke evacuation procedure in response to the energy instrument applying the intraoperative energy.

28-39. (canceled)
